(19) United States
(12) Patent Application Publication
(10) Pub. No.: US 2021/0299156 A1
(43) Pub. Date: Sep. 30, 2021

(54) METHODS OF INHIBITING OR TREATING CORONAVIRUS INFECTION, AND METHODS FOR DELIVERING AN ANTI-NUCLEOLIN AGENT

(71) Applicant: University of Louisville Research Foundation, Inc., Louisville, KY (US)

(72) Inventors: Paula J. Bates, Louisville, KY (US); Kenneth E. Palmer, Anchorage, KY (US); Wajdi Abdul-Ahad, Dana Point, CA (US); Michael Poirier, Vista, CA (US)

(73) Assignee: Qualigen Inc., Carlsbad, CA (US)

(21) Appl. No.: 17/216,528

(22) Filed: Mar. 29, 2021

Related U.S. Application Data
(60) Provisional application No. 63/078,675, filed on Sep. 15, 2020.
(60) Provisional application No. 63/001,308, filed on Mar. 28, 2020.

(51) Int. Cl. A61K 31/711; A61K 45/06; A61K 47/69; A61K 47/10; A61P 31/14

(52) U.S. Cl. CPC A61K 31/711; A61K 45/06; A61P 31/14; A61K 47/10; A61K 47/6929

(57) ABSTRACT

A method of administering an anti-nucleolin agent to a patient by delivering the anti-nucleolin agent to the mouth, lungs, throat, nose and eyes of a patient to prevent COVID-19. Compositions for administering an anti-nucleolin agent comprising a container and a formulation including an anti-nucleolin agent. The anti-nucleolin agent may be administered via an inhaler, nasal spray or eye drop.

### INCORPORATION OF SEQUENCE LISTING BY REFERENCE

The sequences present in this application are provided in an ASCII file named “LOU01-039-US SEQUENCE LISTING.txt”. The ASCII text file is 9,646 bytes, and the date of creation is Jun. 1, 2021. This application incorporates the ASCII text file by reference.

### BACKGROUND

Nucleolin [8] is an abundant, non-ribosomal protein of the nucleolus, the site of ribosomal gene transcription and packaging of pre-ribosomal RNA. This 710 amino acid phosphoprotein has a multi-domain structure consisting of a histone-like N-terminus, a central domain containing four RNA recognition motifs and a glycine/arginine-rich C-terminus, and has an apparent molecular weight of 110 kD. While nucleolin is found in every nucleated cell, the expression of nucleolin on the cell surface has been correlated with the presence and aggressiveness of neoplastic cells [3].

The correlation of the presence of cell surface nucleolin with neoplastic cells has been used for methods of determining the neoplastic state of cells by detecting the presence of nucleolin on the plasma membranes [3]. This observation has also provided new cancer treatment strategies based on administering compounds that specifically target nucleolin [4].

Nucleic acid aptamers are short synthetic oligonucleotides that fold into unique three-dimensional structures that can be recognized by specific target proteins. Thus, their targeting mechanism is similar to monoclonal antibodies, but they may have substantial advantages over these, including more rapid clearance in vivo, better tumor penetration, non-immunogenicity, and easier synthesis and storage.

Guanosine-rich oligonucleotides (GROs) designed for triple helix formation are known for binding to nucleolin. This ability to bind nucleolin has been suggested to cause their unexpected ability to effect antiproliferation of cultured prostate carcinoma cells [6]. The antiproliferative effects are not consistent with a triplex-mediated or an antisense mechanism, and it is apparent that GROs inhibit proliferation by an alternative mode of action. It has been surmised that GROs, which display the propensity to form higher order structures containing G-quartets, work by an aptamer mechanism that entails binding to nucleolin due to a shape-specific recognition of the GRO structure; the binding to cell surface nucleolin then induces apoptosis. The antiproliferative effects of GROs have been demonstrated in cell lines derived from prostate (DU145), breast (MDA-MB-231, MCF-7), or cervical (HeLa) carcinomas and correlates with the ability of GROs to bind cell surface nucleolin [6].

AS1411, a GRO nucleolin-binding DNA aptamer that has antiproliferative activity against cancer cells with little effect on non-malignant cells, was previously developed. AS1411 uptake appears to occur by macropinocytosis in cancer cells, but by a nonmacropinocytic pathway in nonmalignant cells, resulting in the selective killing of cancer cells, without affecting the viability of nonmalignant cells [9]. AS1411 was the first anticancer aptamer tested in humans and results from clinical trials of AS1411 (including Phase II studies in patients with renal cell carcinoma or acute myeloid leukemia) indicate promising clinical activity with no evidence of serious side effects. Despite a few dramatic and durable clinical responses, the overall rate of response to AS1411 was low, possibly due to the low potency of AS1411.

Anti-nucleolin agents conjugated to particles, such as aptamers conjugated to gold nanoparticles, including AS1411 conjugated to gold nanoparticles, have an antiproliferative effect on cancer and tumors [1]. Aptamer conjugated gold nanoparticles, in particular, have a similar or greater antiproliferative effect than the aptamer (anti-nucleolin oligonucleotide) alone, demonstrating similar effects at only 1/10 to 1/100 the dosage. Furthermore, these same agents, preferably having a fluorescent dye conjugated to the particle or attached to the anti-nucleolin agent, may also be used as imaging agents, both in vivo and ex vivo. PEGylation of Aptamer, including AS1411, has been used to improve the circulatory half-life. A detailed review of AS1411 as a cancer-targeting agent, including uses and mechanisms may be found in [2].

Aptamers have been studied as diagnostic tools and anti-viral agents for human viruses [5]. In particular, AS1411 has been used to target host cell nucleolin to treat infections of the human respiratory syncytial virus (RSV) [10]. AS1411 has also been reported to inhibit HIV-1 and Dengue. Nucleolin has been implicated in mediating the uptake, nuclear trafficking and infectiousness of diverse viruses, including coronaviruses [11].

There are no approved treatments to prevent or treat COVID-19, the disease that is caused by SARS-CoV-2. This virus has caused a pandemic that has led to more than 415,000 confirmed infections and more than 18,500 deaths, as of Mar. 24, 2020. Unfortunately, this virus continues to spread globally and is projected to cause millions of deaths if no effective interventions are found and used. Vaccines and new drugs are being developed but their efficacies are still uncertain, and they will take at least a year or more to become available. An effective drug that is already known to be safe for human use would provide an ideal treatment.

Syncytium (syncytia if plural) refers to a multinucleate cell which can result from multiple cell fusions of uninuclear cells. Syncytia can form when cells are infected with certain types of viruses, notably HSV-1, HIV, MeV, and pneumoviruses, for example respiratory syncytial virus (RSV). These syncytial formations create distinctive cytopathic effects when seen in permissive cells. Because many cells fuse together, syncytium are also known as multinucleated giant cells, or polykaryocytes. During infection, viral fusion proteins used by the virus to enter the cell are transported to the cell surface, where they can cause the host cell membrane to fuse with neighboring cells [19].

Cytopathic effect or cytopathogenic effect (abbreviated CPE) refers to structural changes in host cells that are caused by viral invasion. The infecting virus causes lysis of the host cell or when the cell dies without lysis due to an inability to reproduce. Both of these effects occur due to CPEs. If a virus causes these morphological changes in the host cell, it is said to be cytopathogenic. Common examples of CPE include rounding of the infected cell, fusion with adjacent cells to form syncytia, and the appearance of nuclear or cytoplasmic inclusion bodies [20].

Viruses typically enter a patient through the orifices of the body, namely the nose, mouth and eyes. These entrances to the body have a mucus membrane, which, along with the skin, serves as the primary barrier between the external world and the interior of the body. Viruses may be inhaled or ingested.

Inhalers are commonly used to deliver a pharmaceutical composition to the lungs of patients. Inhalers may be used to deliver a drug that acts locally or systematically. There are three main types of inhalers: metered-dose inhalers (MDIs), dry powder inhalers (DPIs) and nebulizers. MDIs use a chemical propellant deliver the medication to the lungs of a patient. Dry powder inhalers (DPIs) deliver medication without using chemical propellants, and are typically activated by patient inhalation. Nebulizers deliver fine liquid mists of medication through a tube or a “mask” that fits over the nose and mouth.

Nasal sprays are commonly used to deliver medications locally or systematically. Many pharmaceutical drugs exist as nasal sprays for systemic administration (for example, treatments for pain, migraine, osteoporosis and nausea). Delivery of systematic medications may be desirable as an agreeable alternative to injection or pills. Nasal sprays are also used to deliver a pharmaceutical composition locally, such as medication to treat allergies.

Eye drops are commonly used to lubricate eyes, but eye drops may also be used to deliver medication. Steroid and antibiotic eye drops are used to treat eye infections, and they may also be used as a prophylactic to prevent infections after eye surgeries.

### SUMMARY

In a first embodiment, the claimed invention includes a composition, comprising: a metering device for pulmonary, nasal or ocular administration, and a formulation inside the metering device, wherein the formulation comprises an anti-nucleolin agent.

In a second embodiment, the claimed invention includes a method of preventing a COVID-19 infection in a patient, comprising: administering an anti-nucleolin agent to a mucus membrane of the patient.

In a third embodiment, the claimed invention includes a method of protecting a mucus membrane of a patient, comprising: administering an anti-nucleolin to the mucus membrane, and re-administering the anti-nucleolin agent to the mucus membrane.

### Definitions

The term “conjugated” means “chemically bonded to”.

The term “anti-nucleolin oligonucleotides” refers to an oligonucleotide that binds to nucleolin.

The term “equivalent aptamer concentration” refers to the concentration of anti-nucleolin oligonucleotide present in a conjugate.

A “pharmaceutically acceptable carrier” includes any and all solvents, dispersion media, coatings, antibacterial and antifungal agents, isotonic and absorption delaying agents which are compatible with pharmaceutical administration. Preferred examples of such carriers or diluents include water, saline, Ringer's solutions and dextrose solution. Supplementary active compounds can also be incorporated into the compositions.

“Medicament,” “therapeutic composition” and “pharmaceutical composition” are used interchangeably to indicate a compound, matter, mixture or preparation that exerts a therapeutic effect in a subject.

“Metering device” refers to any container or device that is designed to provide a controlled dose of medication to a patient. Preferably the device is suitable for pulmonary, nasal or ocular administration.

### BRIEF DESCRIPTION OF THE DRAWINGS

FIG. 1 is two images showing VERO E6 cells incubated with or without SARS-CoV-2.

FIG. 2 is images showing the ability of various concentrations of AS1411 to inhibit SARS-CoV-2-induced cytopathic effects.

FIG. 3 illustrates a metered dose inhaler device.

FIG. 4A illustrates a dry powder inhaler device.

FIG. 4B illustrates a process for forming and dispensing a dry powder formulation.

FIG. 5 illustrates a nebulizer device.

FIG. 6 illustrates a nasal spray device.

FIG. 7 illustrates an eye drop device.

FIG. 8 illustrates a graph of the cell viability as the concentration of aptamer increases.

FIG. 9 illustrates a graph of the virus concentration over time in bronchial epithelium tissue.

FIG. 10 illustrates a graph of the virus concentration over time in human nasal tissue.

FIG. 11 illustrates a graph showing the virus titer for various aptamer concentrations and various time periods in EPI-AIRWAY™ tissue.

FIG. 12 illustrates a graph showing the virus titer for various aptamer concentrations and various time periods in EPI-NASAL™ tissue.

FIG. 13 illustrates a graph showing the percentage of K18-hACE2 mice that survived after each day for 10 days following a SARS-COV-2 infection.

FIG. 14 illustrates a graph showing the weight loss as a percentage of the base weight of the mice for each day for 10 days following a SARS-COV-2 infection.

FIG. 15 illustrates a graph showing the clinical presentation score of the mice for each day for 10 days following a SARS-COV-2 infection.

FIG. 16 illustrates graph showing the percent of cell viability for Vero E6 cells following a SARS-COV-2 virus challenge and various concentrations of AS1411.

FIG. 17 illustrates a graph showing the percent of cell viability for Vero E6 cells following a SARS-COV-2 virus challenge and various concentrations of AS1411, where the AS1411 is administered before the challenge (PrEP) and after the challenge (PEP).

FIG. 18 illustrates a graph showing the percent of cell viability for Vero E6 cells following a SARS-COV-2 virus challenge and various concentrations of AS1411 and various high concentrations of remdesivir.

FIG. 19 illustrates a graph showing the percent of cell viability for Vero E6 cells following a SARS-COV-2 virus challenge and various concentrations of AS1411 and various low concentrations of remdesivir.

FIG. 20 illustrates a graph showing the percent of cell viability for Vero E6 cells following a SARS-COV-2 virus challenge and various concentrations of AS1411 and various high concentrations of mucin.

FIG. 21 illustrates a graph showing the percent of cell viability for Vero E6 cells following a SARS-COV-2 virus challenge and various concentrations of AS1411 and various low concentrations of mucin.

FIG. 22 illustrates a graph showing the percent of cell viability for Vero E6 cells following a SARS-COV-2 virus challenge and various concentrations of AS1411 and various high concentrations of serum.

FIG. 23 illustrates a graph showing the percent of cell viability for Vero E6 cells following a SARS-COV-2 virus challenge and various concentrations of AS1411 and various low concentrations of serum.

FIG. 24 illustrates a graph showing the percent of cell viability for Vero E6 cells following a SARS-COV-2 virus challenge and various concentrations of AS1411 and various high concentrations of dexamethasone.

FIG. 25 illustrates a graph showing the percent of cell viability for Vero E6 cells following a SARS-COV-2 virus challenge and various concentrations of AS1411 and various low concentrations of dexamethasone.

FIG. 26 illustrates a graph showing the TCID₅₀/ml of EPIAIRWAY™ tissue for various concentrations of AS1411.

FIG. 27 illustrates a bar graph showing the TCID₅₀/ml of EPIAIRWAY™ tissue to which the SARS-CoV-2 virus and various concentrations of AS1411 and remdesivir alone and in combination were administered.

FIG. 28 illustrates a bar graph showing the TCID₅₀/ml of EPIAIRWAY™ tissue to which the SARS-CoV-2 virus and various concentrations of AS1411 and dexamethasone alone and in combination were administered.

FIG. 29 illustrates a graph showing the TCID₅₀/ml of EPIAIRWAYυ tissue over time for groups receiving AS1411 and control groups.

FIG. 30 illustrates a graph showing the TCID₅₀/ml of lung tissue from sacrificed mice.

FIG. 31 illustrates a graph showing the TCID₅₀/ml of nasal turbinate tissue from sacrificed mice.

FIG. 32 illustrates a graph showing the TCID₅₀/ml of brain tissue from sacrificed mice.

FIG. 33 illustrates a graph showing the TCID₅₀/ml of kidney tissue from sacrificed mice.

### DETAILED DESCRIPTION

Through experimental work, it has been determined that anti-nucleolin agents are effective at treating and preventing SARS-COV-2 infection, and therefore may be used to treat COVID-19. In particular, AS1411, and anti-nucleolin aptamer, inhibits the action of SARS-COV-2. Other anti-nucleolin agents, including other aptamer, antibodies, and nucleolin targeting proteins, may also be used to treat or prevent SARS-COV-2 infection and treat COVID-19. In addition, anti-nucleolin may optionally be conjugated to nanoparticles and/or PEGylated to have a longer half-life in circulation than some anti-nucleolin agents, in particular aptamers such as AS1411. AS1411 has been tested in more than 100 patients as an anti-cancer agent without any evidence of severe adverse effects. Therefore, it could potentially be moved quickly to use in humans for anti-COVID-19 activity.

Anti-nucleolin agents may also be used to treat other coronavirus infections, other than those caused by SARS-COV-2. Other coronavirus viruses include Severe Acute Respiratory Syndrome (SARS) virus, Middle East Respiratory Syndrome Coronavirus (MERS-CoV), SARS-COV-2 as well as other coronaviruses such as those that cause the common cold.

It may be desirable to administer anti-nucleolin agents to the mouth, nose, lungs and eyes of a patient to prevent a SARS-COV-2 infection. The mouth, nose, lungs and eyes are common locations that viruses enter the body, and cause a viral infection. After administration, the anti-nucleolin agent will remain present in the mouth, nose, lungs or eyes of a patient, in order to inhibit the action of SARS-COV-2. Administering the anti-nucleolin agent to protect the mucus membrane allows for lower effective doses by providing the anti-nucleolin agent at the locations where viral infections begin. These alternative drug delivery routes are also desirable because they avoid the harsh conditions of the gastrointestinal tract, and they are non-invasive and convenient. By inhibiting SARS-COV-2 at the location viral infections typically begin, the anti-nucleolin agent prevents COVID-19 infections. The anti-nucleolin agents may be delivered using an inhaler, nasal spray and/or eye-drops. By administering the anti-nucleolin agent directly to the mouth, nose, lungs or eyes, the anti-nucleolin agent avoids the first-pass metabolism. Administering the anti-nucleolin agent directly to the mouth, nose, lungs or eyes of a patient avoids polymerization or folding of the anti-nucleolin agent, or the binding of the anti-nucleolin agent to blood proteins, which may inactivate the anti-nucleolin agent, as well as rapid clearance by the kidneys. For example, if the anti-nucleolin nucleolin agent is an aptamer, and is circulating in the blood, the aptamer may be polymerized or folded or it may bind to blood proteins and become inactive.

The administration of an anti-nucleolin agent may be carried out using a drug delivery system. A drug delivery system may include an inhaler, nasal sprayer and/or eye-dropper.

Anti-nucleolin agents include (i) aptamers, such as GROs; (ii) anti-nucleolin antibodies; and (iii) nucleolin targeting proteins. Examples of anti-nucleolin agents are found in U.S. Pat. No. 9,452,219 to Bates et al. [12]. Examples of aptamers include guanosine-rich oligonucleotides (GROs). Examples of suitable oligonucleotides and assays are also given in Miller et al. [7]. Characteristics of GROs include:

(1) having at least 1 GGT motif,

(2) preferably having 4-100 nucleotides, although GROs having many more nucleotides are possible,

(3) optionally having chemical modifications to improve stability.

Especially useful GROs form G-quartet structures, as indicated by a reversible thermal denaturation/renaturation profile at 295 nm [6]. Preferred GROs also compete with a telomere oligonucleotide for binding to a target cellular protein in an electrophoretic mobility shift assay [6]. In some cases, incorporating the GRO nucleotides into larger nucleic acid sequences may be advantageous; for example, to facilitate binding of a GRO nucleic acid to a substrate without denaturing the nucleolin-binding site. Examples of oligonucleotides are shown in Table 1; preferred oligonucleotides include SEQ IDs NOs: 1-7; 9-16; 19-30 and 31 from Table 1.

TABLE 1
Non-antisense GROs that bind nucleolin and non-binding controls^{1,2,3}.

| GRO | Sequence | SEQ ID NO: |
|---|---|---|
| GRO29A¹ | tttggtggtg gtggttgtgg tggtggtgg | 1 |
| GRO29-2 | tttggtggtg gtggttftgg tggtggtgg | 2 |
| GRO29-3 | tttggtggtg gtggtggtgg tggtggtgg | 3 |
| GRO29-5 | tttggtggtg gtggtttggg tggtggtgg | 4 |
| GRO29-13 | tggtggtggt ggt | 5 |
| GRO14C | ggtggttgtg gtgg | 6 |
| GRO15A | gttgtttggg gtggt | 7 |
| GRO15B² | ttgggggggg tgggt | 8 |
| GRO25A | ggttggggtg ggtggggtgg gtggg | 9 |
| GRO26B¹ | ggtggtggtg gttgtggtgg tggtgg | 10 |
| GRO28A | tttggtggtg gtggttgtgg tggtggtg | 11 |
| GRO28B | tttggtggtg gtggtgtggt ggtggtgg | 12 |
| GRO29-6 | ggtggtggtg gttgtggtgg tggtggttt | 13 |
| GRO32A | ggtggttgtg gtggttgtgg tggttgtggt gg | 14 |
| GRO32B | tttggtggtg gtggttgtgg tggtggtggt tt | 15 |
| GRO56A | ggtggtggtg gttgtggtgg tggtggttgt | 16 |
| | ggtggtggtg gttgtggtgg tggtgg | |
| CRO | cctcctcctc cttctcctcc tcctcc | 17 |
| CRO-2 | tttcctcctc ctccttctcc tcctcctcc | 18 |
| GRO A | ttagggttag ggttagggtt aggg | 19 |
| GRO B | ggtggtggtg g | 20 |
| GRO C | ggtggttgtg gtgg | 21 |
| GRO D | ggttggtgtg gttgg | 22 |
| GRO E | gggttttggg | 23 |
| GRO F | ggttttggtt ttggttttgg | 24 |
| GRO G1 | ggttggtgtg gttgg | 25 |
| GRO H1 | ggggttttgg gg | 26 |
| GRO I1 | gggttttggg | 27 |
| GRO J1 | ggggttttgg ggttttgggg ttttgggg | 28 |
| GRO K1 | ttggggttgg ggttggggtt gggg | 29 |
| GRO L1 | gggtgggtgg gtgggt | 30 |
| GRO M1 | ggttttggtt ttggttttgg ttttgg | 31 |
| GRO N2 | tttcctcctc ctccttctcc tcctcctcc | 32 |
| GRO O2 | cctcctcctc cttctcctcc tcctcc | 33 |
| GRO P2 | tggggt | 34 |
| GRO Q2 | gcatgct | 35 |
| GRO R2 | gcggtttgcg g | 36 |
| GRO S2 | tagg | 37 |
| GRO T2 | ggggttgggg tgtggggttg ggg | 38 |

¹Indicates a good plasma membrane nucleolin-binding GRO.
²Indicates a nucleolin control (non-plasma membrane nucleolin binding).
³GRO sequence without ¹ or ² designations have some anti-proliferative activity.

Any antibody that binds nucleolin may also be used. In certain instances, monoclonal antibodies are preferred as they bind single, specific and defined epitopes. In other instances, however, polyclonal antibodies capable of interacting with more than one epitope on nucleolin may be used. Many anti-nucleolin antibodies are commercially available, and are otherwise easily made. See, for example, US Patent Application Publication No. US 2013/0115674 to Sutkowski et al. Table 2 lists a few commercially available anti-nucleolin antibodies.

TABLE 2
commercially available anti-nucleolin antibodies

| Antibody | Source | Antigen source |
|---|---|---|
| p7-1A4 Mouse monoclonal antibody | Developmental Studies | Xenopus laevis oocytes |
| (mAb) | Hybridoma Bank | |
| Sc-8031 mouse mAb | Santa Cruz Biotech | human |
| Sc-9893 goat polyclonal Ab (pAb) | Santa Cruz Biotech | human |
| Sc-9892 goat pAb | Santa Cruz Biotech | human |
| Clone 4E2 mouse mAb | MBL International | human |
| Clone 3G4B2 mouse mAb | Upstate Biotechnology | dog |
| | | (MDCK cells) |
| Nucleolin, Human (mouse mAb) | MyBioSource | human |
| Purified anti-Nucleolin-Phospho, | Bio Legend | human |
| Thr76/Thr84 (mouse mAb) | | |
| Rabbit Polyclonal Nucleolin Antibody | Novus Biologicals | human |
| Nucleolin (NCL, C23, FLJ45706, | US Biological | human |
| FLJ59041, Protein C23) Mab Mo xHu | | |
| Nucleolin (NCL, Nucl, C23, FLJ45706, | US Biological | human |
| Protein C23) Pab Rb xHu | | |
| Mouse Anti-Human Nucleolin Phospho- | Cell Sciences | human |
| Thr76/Thr84 Clone 10C7 mAb | | |
| Anti-NCL/Nucleolin (pAb) | LifeSpan Biosciences | human |
| NCL purified MaxPab mouse polyclonal | Abnova | human |
| antibody (B02P) | | |
| NCL purified MaxPab rabbit polyclonal | Abnova | human |
| antibody (D01P) | | |
| NCL monoclonal antibody, clone 10C7 | Abnova | human |
| (mouse mAb) | | |
| Nucleolin Monoclonal Antibody (4E2) | Enzo Life Sciences | human |
| (mouse mAb) | | |
| Nucleolin, Mouse Monoclonal Antibody | Life Technologies | human |
| | Corporation | |
| NCL Antibody (Center E443) (rabbit | Abgent | human |
| pAb) | | |
| Anti-Nucleolin, clone 3G4B2 (mouse | EMD Millipore | human |
| mAb) | | |
| NCL (rabbit pAb) | Proteintech Group | human |
| Mouse Anti-Nucleolin Monoclonal | Active Motif | human |
| Antibody, Unconjugated, Clone 3G4B20 | | |
| Nsr1p - mouse monoclonal | EnCor Biotechnology | human |
| Nucleolin (mouse mAb) | Thermo Scientific Pierce | human |
| | Products | |
| Nucleolin [4E2] antibody (mouse mAb) | GeneTex | human |

Nucleolin targeting proteins are proteins, other than antibodies, that specifically and selectively bind nucleolin. Examples include ribosomal protein S3, tumor-homing F3 peptides [44,45] and myosin H9 (a non-muscle myosin that binds cell surface nucleolin of endothelial cells in angiogenic vessels during tumorigenesis).

Anti-nucleolin agents may optionally be conjugated to particles made of a variety of materials solid materials, including (1) metals and elements; (2) oxides; (3) semiconductors; and (4) polymers. Metals and elements, preferably non-magnetic metals and elements, include gold, silver, palladium, iridium, platinum and alloys thereof; elements include silicon, boron and carbon (such as diamond, graphene and carbon nanotubes), and solid compounds thereof. Oxides include titanium dioxide, silicon dioxide, zinc oxide, iron oxide, zirconium oxide, magnesium oxide, aluminum oxide and complex oxides thereof, such as barium titanate. Semiconductors include quantum dots, zinc sulfide, silicon/germanium alloys, boron nitride, aluminum nitride, and solid solutions thereof. Polymers include polyethylenes, polystyrenes, polyacrylamide, polyacrylates and polymethacrylates, and polysiloxanes. Preferably, the particles are non-toxic. The particles are preferably nanoparticles having an average particle diameter of 1-100 nm, more preferably 1-50 nm, including 4, 5, 6, 7, 8, 9, 10, 12, 15, 18, 20, 25, 30, 35, 40, 45, 50, 55, 60, 65, 70, 75, 80, 85, 90 and 95 nm.

Oligonucleotides and proteins have been attached to solid materials, such as metals and elements, oxides, semiconductors and polymers, by a variety of techniques. These same techniques may be used to attached anti-nucleolin agents to particles. Particularly preferred compositions are aptamers conjugated to gold nanoparticles. Gold nanoparticles (GNPs) exhibit low toxicity, versatile surface chemistry, light absorbing/scattering properties, and tunable size. Aptamers effectively cap gold particles and prevent aggregation, are safe, stable, easy to synthesize, and non-immunogenic.

The amounts and ratios of compositions described herein are all by weight, unless otherwise stated. Accordingly, the number of anti-nucleolin agents per nanoparticle may vary when the weight of the nanoparticle varies, even when the equivalent anti-nucleolin agent concentration (or equivalent aptamer concentration) is otherwise the same. For example, the number of anti-nucleolin agent molecules per nanoparticle may vary from 2 to 10,000, or 10 to 1000, including 20, 30, 40, 50, 60, 70, 80, 90, 100, 200, 300, 400, 500, 600, 700, 800 and 900.

Optionally, the anti-nucleolin agent may be conjugated to PEG. Any molecular weight of PEG may be used. Preferably, the PEG molecular weight is 200 to 20,000, more preferably 1000 to 10,000, including 2000, 3000, 4000, 5000, 6000, 7000, 8000 and 9000, and values in between. It is understood that these molecular weight values are number-average molecular weights that represent the value as expressed to the significant figures of the accuracy of the measurement, and that such compositions have a distribution of PEG molecules about the reported molecular weight.

Optionally, the anti-nucleolin agent may be conjugated to both PEG and nanoparticle. The molar ratio between the PEG and anti-nucleolin agent conjugated to the nanoparticles may be varied. For example, the ratio of anti-nucleolin agent:PEG may be 1:1, 2:1, 3:1, 4:1, 5:1, 6:1, 7:1, 8:1, 9:1, 10:1, 11:1 or 12:1. Alternatively, the ratio of PEG:anti-nucleolin agent may be 1:1, 2:1, 3:1, 4:1, 5:1, 6:1, 7:1, 8:1, 9:1, 10:1, 11:1 or 12:1. Preferably, the anti-nucleolin agent is AS1411 (SEQ ID NO:10). Preferably, the nanoparticles are gold nanoparticles having a diameter of 4, 5, 10, 15, 20, or 100 nm.

When conjugating an anti-nucleolin agent (such as AS1411) and/or PEG to a nanoparticle (such as gold nanoparticles), typically the surface of the nanoparticle is saturated with the agents conjugated to the surface. Therefore, when the mixture of agents (such as AS1411 and PEG) is formed, the ratio of the agents is controlled, and this controls the ratio of the agents conjugated to the nanoparticles, and the number of each agent on each nanoparticle. For example, in the case of 4 nm gold nanoparticles conjugated with AS1411 and PEG, the total number of agent molecules which are conjugated will be 12, so the sum of AS1411 and PEG molecules conjugated to the nanoparticle will be 12. The nomenclature used to identify the ratio of AS1411:PEG is based on the total number of AS1411 molecules conjugated to the nanoparticle. In the case of a 4 nm gold nanoparticle, “12X” means 12 AS1411 molecules, and an AS1411:PEG ratio of 12:0; similarly, “9X”, “6X”, “3X” and “0X”, have 9, 6, 3 and 0 AS1411 molecules, respectively, and a AS1411:PEG ratio of 9:3, 6:6, 3:9 and 0:12, respectively (or alternatively 3:1, 1:1, 1:3 and 0, respectively). With a larger gold nanoparticle, such as a 5 nm or 10 nm gold nanoparticle, the total amount agents which may be conjugated is greater, so “9X” would imply a smaller AS1411:PEG ratio. Although a distribution of conjugates form during synthesis, those nanoparticles which deviate from the most prevalent conjugation ratio may be separated by “salting out,” taking advantage of the different zeta potentials of nanoparticles with different conjugation ratios.

Anti-nucleolin agents may be used to formulate a pharmaceutical composition for treating or preventing SARS-COV-2 infections, by forming mixtures of the anti-nucleolin agent and a pharmaceutically acceptable carrier, such as a pharmaceutical composition. Methods of treating or preventing SARS-COV-2 infections in a subject include administering a therapeutically effective amount of an anti-nucleolin agent.

A pharmaceutical composition is formulated to be compatible with its intended route of administration, including intravenous, intradermal, subcutaneous, oral, inhalation, transdermal, transmucosal, and rectal administration, as well as by eye drop and nasal spray. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide.

An inhaler may be used to deliver an anti-nucleolin agent to the lungs of a patient. The inhaler may also deliver the anti-nucleolin agent to the mucus membrane of the mouth and/or throat of the patient. Inhalers include metered-dose inhalers (MDI), dry-powder inhalers (DPI) and nebulizers. A nasal spray may be used to deliver an anti-nucleolin agent to the nasal passage, via a patient's nose. Eye-drops containing an anti-nucleolin agent may be administered to the eyes of a patient.

MDIs include the metering device and a formulation inside the metering device. The metering device of the MDI may include a container (such as, an aluminum can or plasticized glass vial), metering valve and actuator. MDIs may optionally include a dose counter to count the number of remaining doses. MDIs may optionally include chambers or spacers to provide better delivery of aerosol and ease of use.

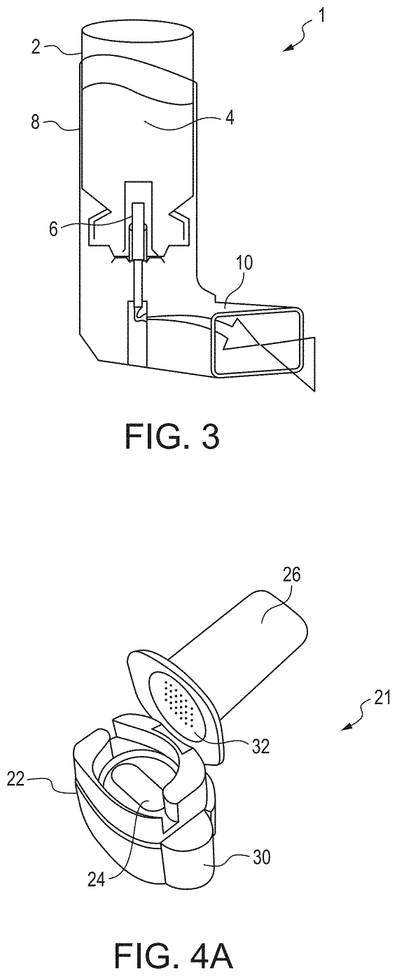

FIG. 3 illustrates an MDI 1, having a container 2, a formulation 4 inside the container, a metering valve 6, and an actuator 8. The actuator 8 is a plastic holder that includes a mouthpiece 10.

The formulation of the MDI includes an anti-nucleolin agent and a chemical propellant to push the anti-nucleolin agent out of the container. The propellant is preferably a liquified compressed gas, which functions as a driving force. Preferably the propellant has the same vapor pressure regardless of when the container is full or nearly empty. The propellant must be toxicologically safe and chemically non-reactive with the anti-nucleolin agent. The propellant may be chlorofluorocarbon (CFC) or hydrofluoroalkanes (HFA). The CFC propellants may be trichlorofluoromethane (CFC 11), dichlorodifluoromethane (CFC 12), dichlorotetrafluoroethane (CFC 114), or blends of these propellants [13]. The HFA may be 1,1,1,2-tetrafluoroethane (HFA 134a) and 1,1,1,2,3,3,3-heptafluoropropane (HFA 227), or 1,1-difluoroethane (HFA 152a). Other possible propellants include propane, n-butane, isobutane, n-pentane, isopentane, neopentane, dimethylether, and hydrofluoro-olefins (HFO). Preferably the propellant is an HFA.

MDIs can be formulated with the drug completely dissolved in the formulation, rendering a solution formulation, or with the drug practically insoluble in the formulation, rendering a suspension formulation. The MDI may optionally include excipients. Solvents and/or excipients may be included to make the anti-nucleolin agent soluble. The solvents and/or excipients may also alter the dissolution of the particles from the aerosol spray in the lungs, which may affect the pharmacological effect of the anti-nucleolin agent. Cosolvents may also be added to the formulation to help increase the solubility of the drug or the other excipients.

The excipients may be an antioxidant, preservative, flavoring agent, chelating agent, cosolvent, humectant, buffering agent, pH adjusting agent, dispersion agent, suspending aid, and emulsifying agent [13]. Approved excipients include acetone, sodium bisulfate, ammonia, ascorbic acid, benzalkonium chloride, cetylpyridinium chloride, chlorobutanol, citric acid (anhydrous), edetate sodium/edetate disodium, ethanol, dehydrated alcohol, alcohol, glycerin, glycine, hydrochloric acid, lecithin (soya), lysine monohydrate, magnesium stearate, menthol, methylparaben, nitric acid, oleic acid, polyethylene glycol 1000, polysorbate 80, polyvinylpyrrolidone K25, propylene glycol, propylparaben, saccharin, saccharin sodium dehydrate, sodium bisulfate, sodium bisulfite, sodium chloride, sodium citrate, sodium hydroxide, sodium metabisulfite, sodium sulfate (anhydrous), sodium sulfite, sorbitan trioleate (Span 85), sulfuric acid, thymol, tromethamine and water. Preferably ethanol is included as a cosolvent. The ethanol concentration may be varied to improve the solubility of the anti-nucleolin agent.

DPIs include the metering device and the dry powder formulation. The metering device may include a powder reservoir, a blister disk, a blister strip, capsules, or other suitable designs for dry powder inhalers. The dry powder formulation may include a dry powder having an anti-nucleolin agent and a carrier particle, which prevents aggregation and improves flow. DPIs may be activated by the patient's inspiratory airflow. When the patient inhales, airflow through the device creates shear and turbulence; air is introduced to the static powder, and the airflow fluidizes the powder and the powder enters the patient's airway.

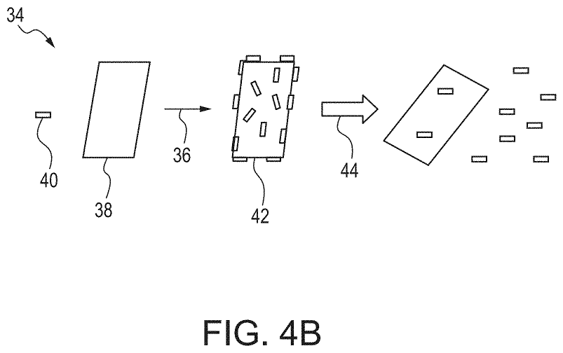

FIG. 4A illustrates a dry powder inhaler 21, which includes a container 22. The container is configured to hold a capsule 24, which will contain the dry powder formulation. The container 22 has a mouthpiece 26. The dry powder inhaler 21 may have a button 30 to break the capsule and a screen 32 to hold the exterior of the capsule in place. The DPI may be a different type of metering device that does not use a capsule to hold the dry powder formulation.

FIG. 4B illustrates a diagram 34 showing the blending process 36 of the carrier particles 38 and the anti-nucleolin agents 40. In the blending process 36, the anti-nucleolin agents 40 are blended with the carrier particles 38 to form the dry powder formulation 42. In the shearing step 44, the anti-nucleolin agents 40 are separated from the carrier particles 38.

Carrier particles possess physio-chemical stability, biocompatibility and biodegradability, and are compatible with the drug substance. During inhalation, the drug particles are detached from the surface of the carrier particles by the energy of the inspired air flow that overcomes the adhesion forces between drug and carrier. The particle size, shape, surface area, and physiochemical properties determine the aerodynamic property of the particle, which impacts the delivery of the anti-nucleolin agent. It is well-known in the art that the size, shape, and surface of the carrier particle may be adjusted to improve the drug delivery properties of the carrier particle. The properties of the carrier particle must balance between providing enough adhesion between drug and carrier to produce a stable formulation (homogeneous mixture with no powder segregation and proper uniformity) while also allowing for easy separation of the drug during inhalation. The carrier particle may be a sugar carrier, such as α-lactose monohydrate, mannitol, trehalose dihydrate, sorbitol, raffinose pentahydrate, maltose monohydrate, dextrose monohydrate, xylitol, erythritol, or sucrose [14]. The carrier particle may be a cyclodextrin such as α-cyclodextrin, β-cyclodextrin, or γ-cyclodextrin. The carrier particle may be a metal stearate such as magnesium stearate, calcium stearate, or zinc stearate. The carrier particle may be an amino acid such as leucine or trileucine.

Anti-nucleolin agents may be delivered using the iSPERSE™ inhaled drug delivery platform (PULMATRIX, Lexington, Mass.). The anti-nucleolin agent may be administered using the delivery system of MANNKIND® Corporation's Technosphere® technology, which uses particles of fumaryl diketopiperazine (FDKP) as the carrier particle (see U.S. Pat. No. 8,227,409).

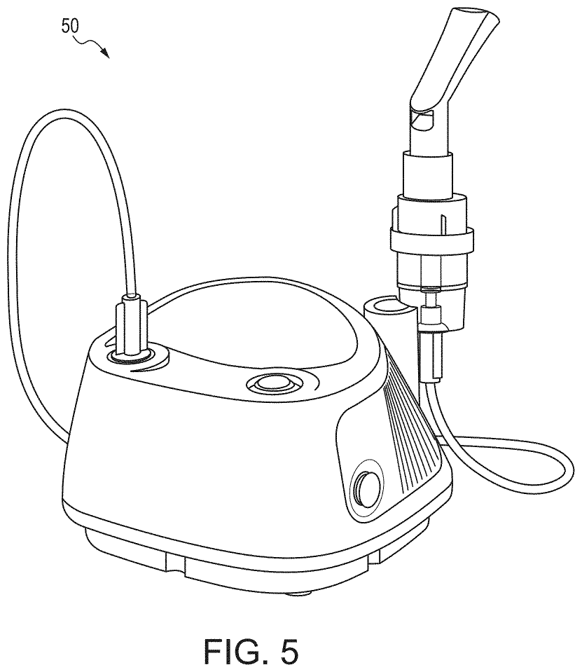

A liquid including an anti-nucleolin agent may be administered using a nebulizer. Nebulizer devices change a liquid pharmaceutical composition to a mist, so it can be inhaled. Nebulizers may be home (tabletop) or portable models. Home nebulizers are larger, and may be plugged into an electrical outlet, and portable nebulizers may run on batteries. To deliver a drug by nebulization, the drug must first be dispersed in an aqueous medium. After application of a dispersing force (either a jet of gas or ultrasonic waves), the drug particles are contained within aerosol droplets, which are then inhaled. Some drugs readily dissolve in water, whereas others need a cosolvent such as ethanol or propylene glycol, or may be delivered as a dispersion. FIG. 5 illustrates a nebulizer device 50. The nebulizer may be an InnoSpire Elegance Nebulizer Compressor.

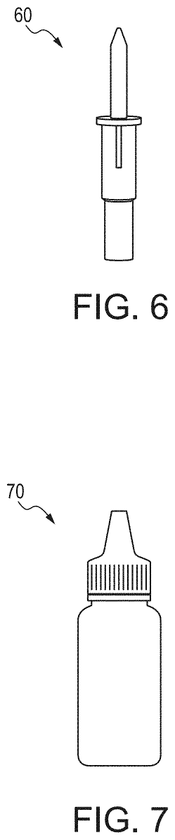

An anti-nucleolin agent may be administered using a nasal spray device. Intranasal drug delivery is recognized to be a useful and reliable alternative to oral and parenteral routes. The nasal route of drug delivery can be used for both local and systemic drug delivery. For instance, localized nasal drug delivery is usually used to treat conditions related to the nasal cavity, such as congestion, rhinitis, sinusitis and related allergic conditions. FIG. 6 illustrates a nasal spray device 60.

The nasal spray formulation may include various excipients such as buffers, solublizers, preservatives, antioxidants, humectants, and surfactants. Examples of buffer used in nasal spray include sodium phosphate, sodium citrate and citric acid. Examples of solubilizers include solvents and cosolvents such as glycols, alcohols, transcutol (diethylene glycol monoethyl ether) and medium chain glycerides. Examples of preservatives include parabens, phenyl ethyl alcohol, benzalkonium chloride, EDTA and benzoyl alcohol. Examples of antioxidants include sodium bisulfite, butylated hydroxytoluene, sodium metabisulfite and tocopherol. Examples of humectants include glycerin, sorbitol and mannitol. The surfactant may be polysorbet.

Anti-nucleolin agents may also be administered by topical ocular administration, that is, eye drop solutions, ointments, in situ gels, inserts, multicompartment drug delivery systems, and ophthalmic drug forms with bioadhesive properties. For example, eye-drop solutions may include an anti-nucleolin agent, and a pharmaceutical carrier. The pH of the eye-drop solution may be adjusted to be suitable for use in ocular administration. The bioavailability of the anti-nucleolin agent may be modified by introducing excipients to formulation, which enhanced drug penetration into the eyeball. These excipients included chelating agents, surfactants, and cyclodextrins, which, along with active ingredients, form inclusion complexes. This increases solubility, permeability, and bioavailability of poorly soluble drugs. FIG. 7 illustrates an eye drop device 70.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, CREMOPHOR EL® (BASF; Parsippany, N.J.) or phosphate buffered saline (PBS). In all cases, the composition must be sterile and should be fluid so as to be administered using a syringe. Such compositions should be stable during manufacture and storage and are preferably preserved against contamination from microorganisms such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as mannitol, sorbitol, and sodium chloride can be included in the composition. Compositions that can delay absorption include agents such as aluminum monostearate and gelatin.

Sterile injectable solutions can be prepared by incorporating the active agents, and other therapeutic components, in the required amount in an appropriate solvent with one or a combination of ingredients as required, followed by sterilization. Methods of preparation of sterile solids include vacuum drying and freeze-drying to yield a solid.

The pharmaceutical composition described herein may further comprise other therapeutically active compounds, and/or may be used in conjunction with physical techniques which are suitable for the treatment or prevention of SARS-COV-2 infections.

In the treatment or prevention of SARS-COV-2 infections, an appropriate dosage level of the therapeutic agent, preferably a GRO such as AS1411, will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 1, 2, 5, 10, 15, 20, 25, 30, 35, 40, 45 or 50 mg/kg per day. In the case of GRO such as AS1411 conjugated to a nanoparticle or PEGylated, the dosages would be equivalent aptamer concentration. The compounds may be administered on a regimen of 1 to 4 times per day, preferably once per day. Administration by continuous infusion is also possible.

Pharmaceutical preparation may be pre-packaged in ready-to-administer form, in amounts that correspond with a single dosage, appropriate for a single administration referred to as unit dosage form. Unit dosage forms can be enclosed in ampoules, disposable syringes or vials made of glass or plastic.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the patient undergoing therapy.

Additional therapeutically active compounds may be administered in combination with an anti-nucleolin agent. The additional therapeutically active compound may be a compound other than an anti-nucleolin agent. The other therapeutically active compound may be selected based on the condition of the patient as well as the patient's medical history. The other therapeutically active compound may operate by targeting the virus or it may operate by treating or preventing Acute Respiratory Distress Syndrome (ARDS) by reducing the patient's immune response, that is the cytokine storm.

The other therapeutically active compound may an antiviral or it may be a compound to address ARDS. Anti-virals include remdesivir, convalescent plasma or serum, interferons, baricitinib, masitinib and combinations thereof. Examples of interferons include beta interferons and lambda interferons. Compounds to address ARDS (anti-ARDS compounds) include dexamethasone, methylprednisolone, famotidine, a combination of famotidine and cetirizine, anti-inflammatory antibodies, and combinations thereof. Examples of anti-inflammation antibodies include abatacept, alefacept, alemtuzumab, atacicept, belimumab, canakinumab, eculizumab, epratuzumab, natalizumab, ocrelizumab, ofatumumab, omalizumab, otelixizumab, rituximab, teplizumab, vedolizumab, adalimumab, briakinumab, certolizumab pegol, etanercept, golimumab, infliximab, mepolizumab, reslizumab, tocilizumab, ustekinumab and combinations thereof.

There may be more than one other therapeutically active compound administered in combination. The number of therapeutically active compounds may be 2, 3, 4, 5, or 6 therapeutically active compounds, including the anti-nucleolin agent such as AS1411. The therapeutically active compound may be provided in a dose that is suitable for the specific compound. The therapeutically active compound will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 1, 2, 5, 10, 15, 20, 25, 30, 35, 40, 45 or 50 mg/kg per day.

### EXAMPLES

### Example 1: Cytopathic Effect Assay Illustrating Anti-SARS-CoV-2 Activity of AS1411

Cells were seeded at a density of 12000 cells/well into 96-well flat-bottomed tissue culture plates, 0.1 ml/well, and incubated overnight at 37° C. Next day, the growth medium was decanted and AS1411 was prepared in Virus growth medium (VGM) from starting concentration of 0.3125 μM to 20 μM and added to each well (3 wells/dilution, 0.1 ml/well). Only VGM was added to cell and virus control wells (0.1 ml/well). SARS-CoV-2 at a multiplicity of infection (MOI) of 0.01, diluted in VGM and added to compound test wells (3 wells/dilution of compound) and to virus control wells at 0.1 ml/well. Virus was added approximately 5 minutes after compound. The plates were incubated at 37° C. in a humidified incubator with 5% CO₂ for 72 hours. After 72 hours, cell viability was measured by Neutral Red Dye uptake assay.

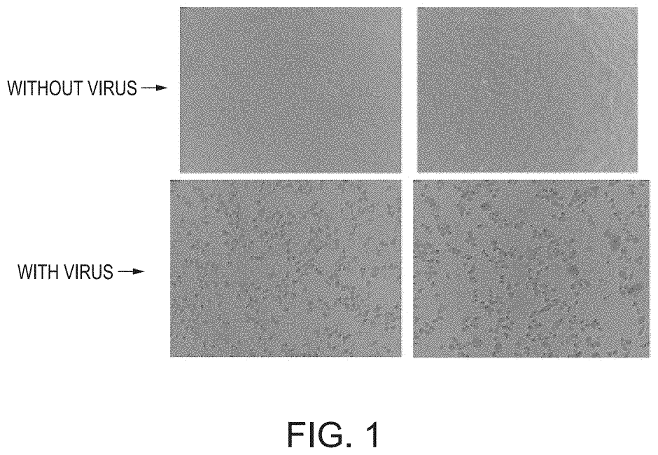

FIG. 1 is two images showing VERO E6 cells incubated with or without SARS-CoV-2. Infection with SARS-CoV-2 induces cytopathic effects including formation of syncytia (large multinucleated cells caused by virus-induced cell fusion).

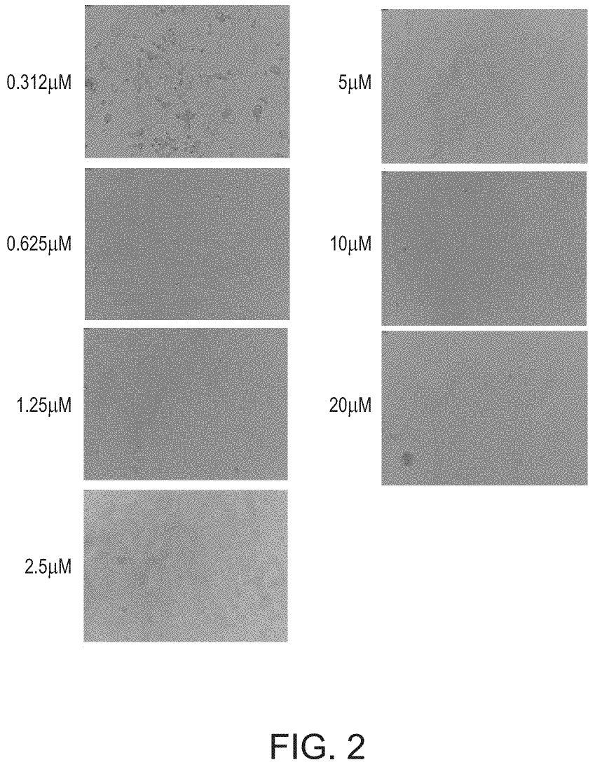

FIG. 2 is images showing the ability of various concentrations of AS1411 to inhibit SARS-CoV-2-induced cytopathic effects. In the presence of AS1411, at doses of 0.625 μM and above, the cytopathic effects of SARS-CoV-2 are markedly reduced and cells appear more like the uninfected (“without virus”) controls.

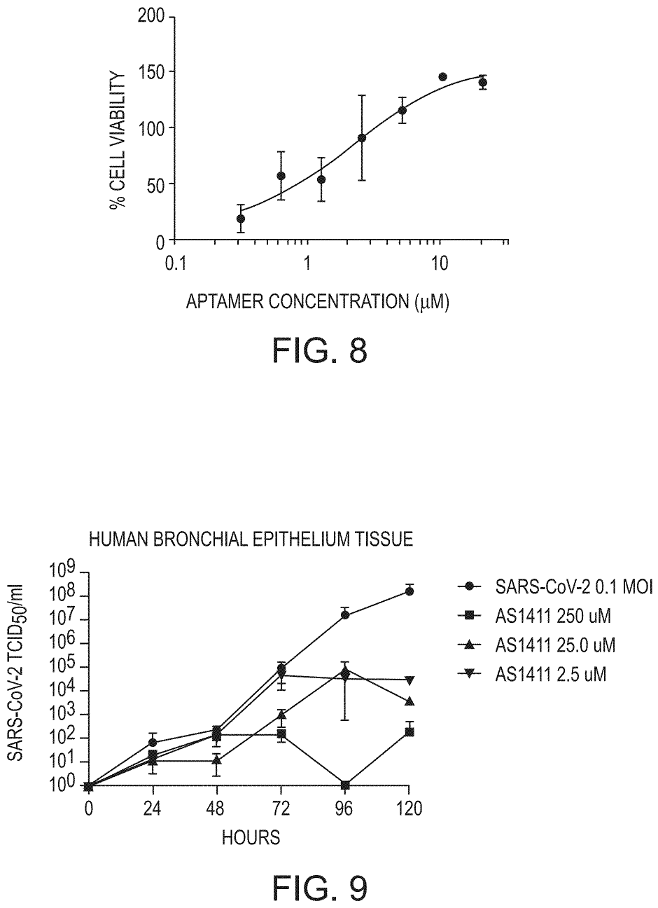

The data indicate that anti-nucleolin agents, such as AS1411, inhibit infection by the novel coronavirus, SARS-CoV-2, as well as other coronaviruses. AS1411 inhibits SARS-CoV-2 virus-induced syncytium formation without cytotoxicity beginning at a concentration 0.625 μM. This is a lower concentration than those concentrations which show an anti-cancer effect. FIG. 8 shows the percent of viable cells as the concentration of AS1411 increases.

### Example 2: 3D Human Bronchial Epithelium Tissues

We have adopted a 3D human bronchial epithelium tissue model in our product development program to assess tissue-specific effects and identify potential safety risks. The information generated in this model will help support the design and conduct of clinical studies.

The EPIAIRWAY™ (MATTEK™ Life Sciences) is a ready-to-use, 3D mucociliary tissue model consisting of normal, human-derived tracheal/bronchial epithelial cells that is also available as a co-culture system with normal human stromal fibroblasts (EpiAirwayFT). Cultured at the air-liquid interface (ALI), EPIAIRWAY™ recapitulates the in vivo phenotypes of barrier, mucociliary responses, infection, toxicity responses and disease. EPIAIRWAY™ is amenable to acute or long-term chronic studies across a wide range of highly predictive in vitro applications.

Replication of SARS-CoV-2 on MATTEK™ human broncho-epithelial airway cultures was examined. The EPIAIRWAY™ tissues were prepared and cultured according to the vendor suggested methods. We pretreated tissues with AS1411 at 2.5 μM, 25 μM and 250 μM final concentrations (corresponding to the planned clinical dose); 0.1× the clinical dose and 0.01× the clinical dose prior to challenge with SARS-CoV-2 at multiplicity of infection (MOI) of 0.1. Progeny virus release at the apical surface was determined by determining the 50% tissue culture infective dose (TCID₅₀) in VeroE6 cells. Triplicate wells were used for each concentration of AS1411 tested.

FIG. 9 shows a significant reduction in viral titer (up to approximately 5 logs fold) in the 3-Dimensional human bronchial epithelium tissue (MATTEK™ EPIAIRWAY™) upon application of AS1411. FIG. 11 shows the reduction in virus titer as the time is increased for various AS1411 concentrations, as well as for virus only, at a MOI of 0.1 and 0.5.

### Example 3: 3D Human Nasal Epithelium Tissues

We have adopted a 3D human nasal epithelium tissue model in our product development program to assess tissue-specific effects and identify potential safety risks. The information generated in this model will help support the design and conduct of clinical studies.

The EPINASAL™ (MATTEK™ Life Sciences) is a prototype ready-to-use, 3D mucociliary tissue model consisting of normal, human-derived nasal epithelial cells. Cultured at the air-liquid interface (ALI), the EPINASAL™ prototype recapitulates the in vivo phenotypes of barrier, mucociliary responses, infection, toxicity responses and disease.

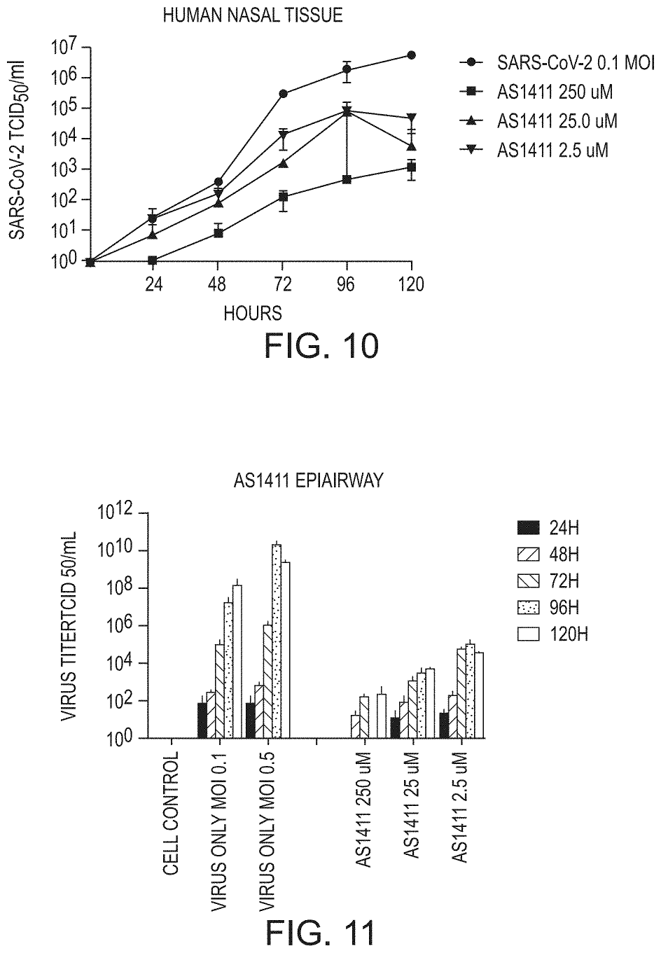

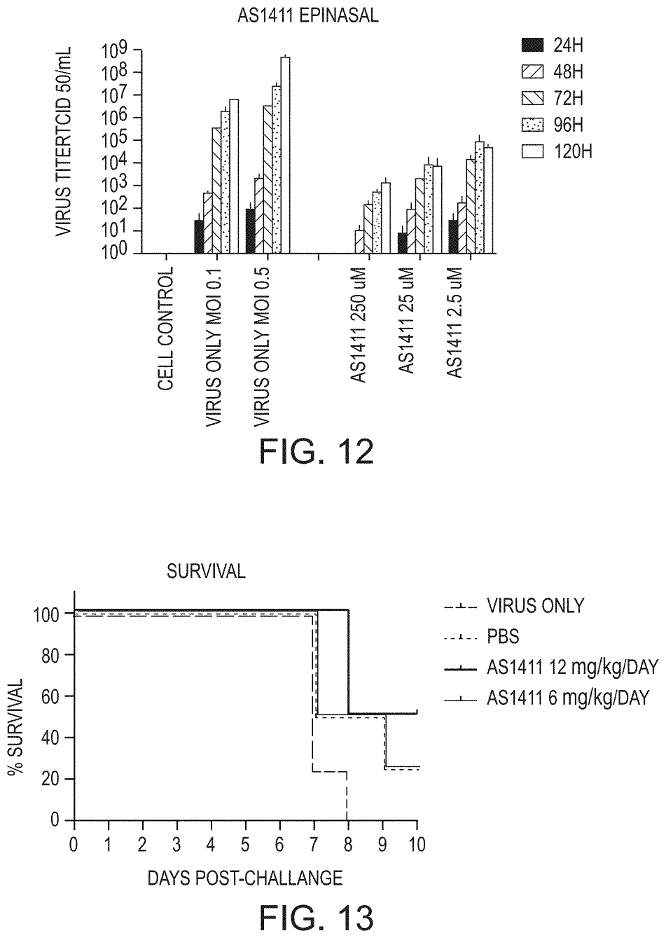

Replication of SARS-CoV-2 on MATTEK™ human nasal tissue cultures was examined. The EPINASAL™ tissues were prepared and cultured according to the vendor suggested methods. We pretreated tissues with AS1411 at 2.5 μM, 25 μM and 250 μM final concentrations (corresponding to the planned clinical dose); 0.1× the clinical dose and 0.01× the clinical dose prior to challenge with SARS-CoV-2 at MOI of 0.1. Progeny virus release at the apical surface was determined by determining the TCID₅₀ in VeroE6 cells. Triplicate wells were used for each concentration of AS1411 tested. FIG. 10 shows a significant reduction in viral titer (up to approximately 3.5 logs fold) in the 3D human nasal epithelium tissue (MATTEK™ EPINASAL™) upon application of AS1411. FIG. 12 shows the reduction in virus titer as the time is increased for various AS1411 concentrations, as well as for virus only, at a MOI of 0.1 and 0.5.

### Example 4: K18-hACE2 Mouse Study

K18-hACE2 mice are an acceptable model of SARS-CoV-2 infection in humans (Moreau et al., “Evaluation of K18-hACE2 Mice as a Model of SARS-CoV-2 Infection”, Am. J. Trop. Med. Hyg., 103(3), pp. 1215-1219 (2020)). This model represents the worst-case scenario with regard to acute respiratory distress syndrome (ARDS) in human COVID-19. Any significant reduction in lethality or clinical symptoms is likely to be much more significant with regards to human treatment.

A study was conducted to examine the effect of AS1411 in 4 groups of K18-hACE2 mice. The 4 groups of mice were given virus only, phosphate-buffered saline (PBS), 12 mg/kg/per day of AS1411, or 6 mg/kg/per day of AS1411. Osmotic pumps (AZLET® pumps model number 1007D) were used for intraperitoneal administration of AS1411 and PBS for 7 days, and were implanted 1 day prior to the virus challenge (day −1). The 12 mg/kg group represents ‘neat’ 20 mg/ml AS1411 dispensed by the AZLET® pump at a rate of 0.5 ul/hr to an average 20 g mouse. The 4 groups of mice were challenged with SARS-CoV-2 at day 0. The “PBS” mice received mock treatment consisting of surgical intraperitoneal implantation with an osmotic pump containing PBS in place of AS1411. The “virus only mice” received no surgery or treatment.

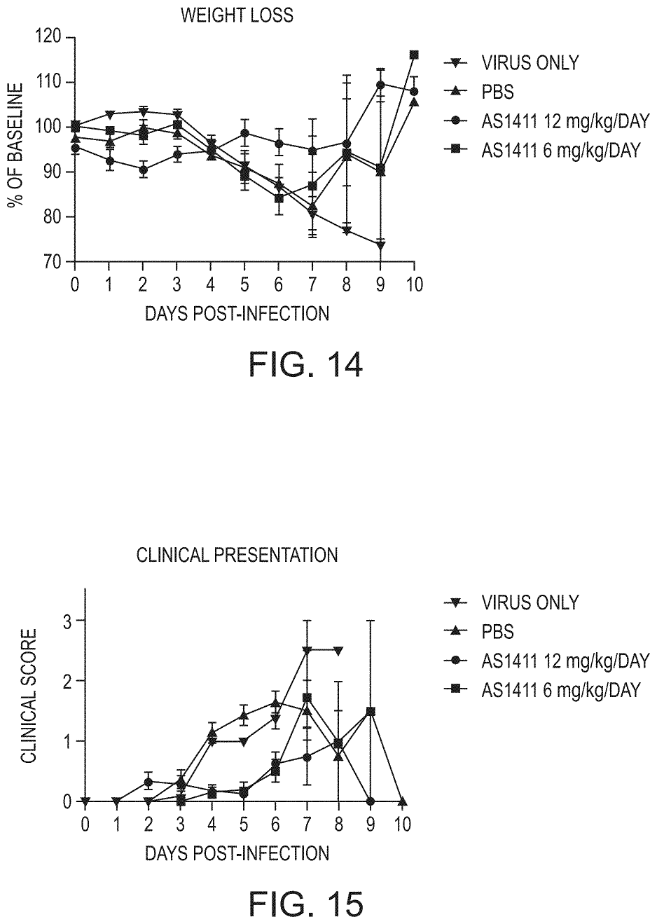

FIG. 13 shows the survival percentage for the mice for each day following the virus challenge. FIG. 14 shows the weight loss as a percent of the baseline weight for the mice. FIG. 15 shows the clinical score of the mice. The clinical score is comprised of two scores taken each day (once in the morning and once in the evening) based on physical appearance, where 0=normal; 1=mild—lack of grooming; 2=moderate—rough coat, hunched posture, moderate dyspnea; and 3=severe—severe respiratory distress, neurologic symptoms (for example, seizures, hind limb paralysis), unresponsive to external stimuli (such mice were euthanized).

7 days after the virus challenge, the mice that received the virus had a survival percentage of only about 25%, while the mice that received 12 mg/kg per day of AS1411 had a 100% survival rate (FIG. 13). 10 days from virus challenge the survival percentage for all 4 groups dropped significantly. As the treatment with AS1411 ended 7 days from implantation (day 6), it may be fair to only consider data up to day 7 post-challenge. In humans administration would continue. The inflammatory response to surgery may explain the difference in outcomes between the virus only group and the PBS group.

### Example 5: Vero E6 In Vitro Study of AS1411 Alone and in Combination with Other Compounds

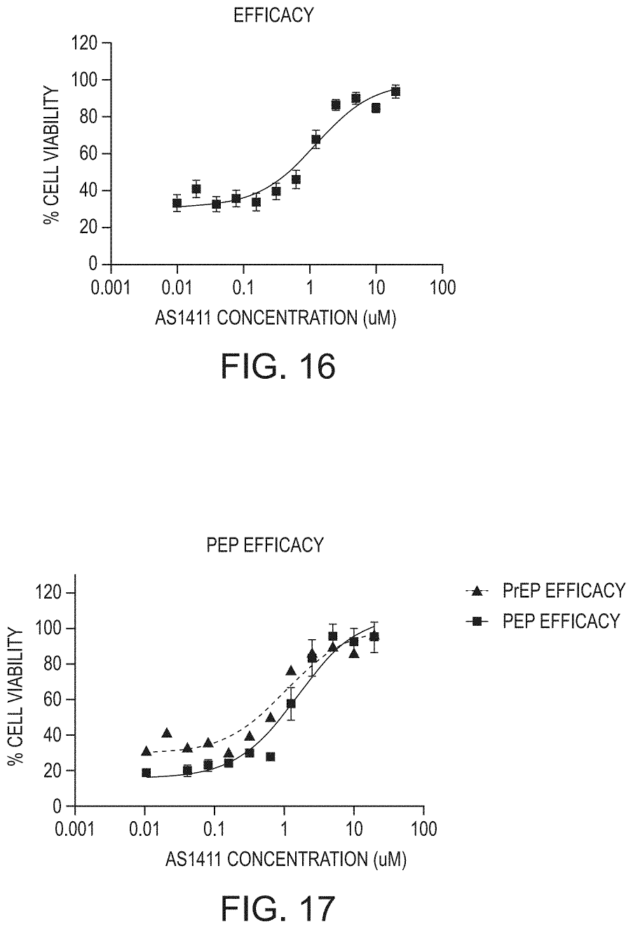

The percentage of cell viability as a function of AS1411 concentration was measured for pre-exposure prophylaxis (PrEP) and post-exposure prophylaxis (PEP). In vitro experiments were conducted on Vero E6 cells by administration of the SARS-CoV-2 virus at multiplicity of infection (MOI) of 0.1 along with AS1411. The AS1411 was provided in various concentrations, from 0.01 μM to 10 μM. For the PrEP and PEP studies, 4 hours elapsed between administration of the drug and the virus challenge. FIG. 16 shows that cell viability is dose dependent for PrEP. FIG. 17 shows that cell viability is dose dependent for PEP as well, and compares the effectiveness of PrEP and PEP. Table 3 shows the EC50 and EC90 of AS1411 when administered before or after the virus challenge.

TABLE 3

| | |
|---|---|
| AS1411 | Concentration (uM) |

| Efficacy | EC50 | EC90 |
|---|---|---|
| PrEP | 0.986 | 2.16 |
| PEP | 1.496 | 3.2 |

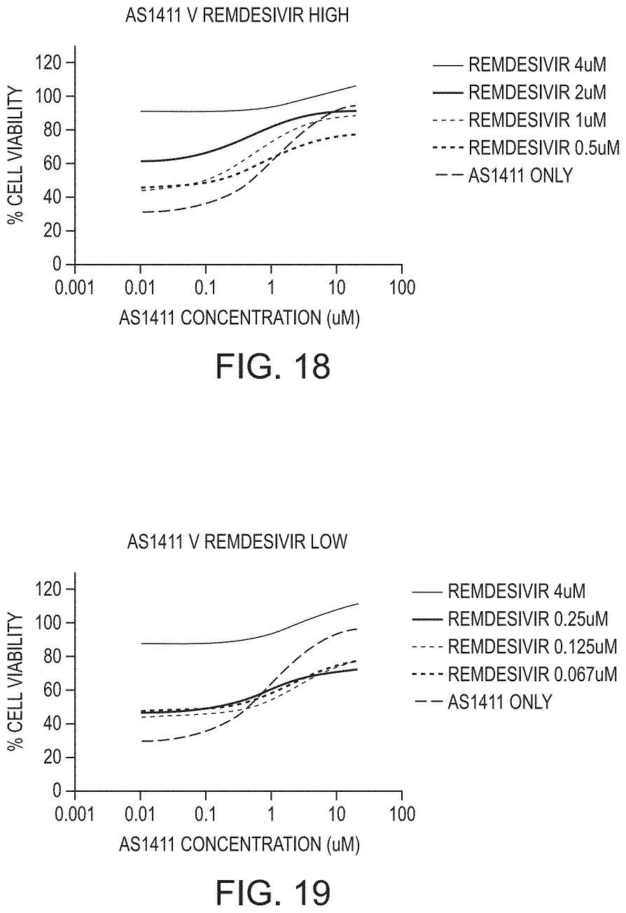

The combination of AS1411 and other compounds or drugs was tested in Vero E6 cells to study the potential drug interference. The treatments were administered to the cells 4 hours after the virus challenge. FIGS. 18 and 19 show the percentage of cell viability for varying concentrations of AS1411 and various concentrations of remdesivir. Table 4 below shows the half maximal effective concentration (EC50) of each remdesivir concentration and AS1411.

TABLE 4

| Remdesivir (uM) + AS1411 | EC50 |
|---|---|
| 4 | 3.435 |
| 2 | 0.4715 |
| 1 | 0.5501 |
| 0.5 | 0.8178 |
| 0.25 | 0.9124 |
| 0.125 | 2.667 |
| 0.0625 | 2.072 |

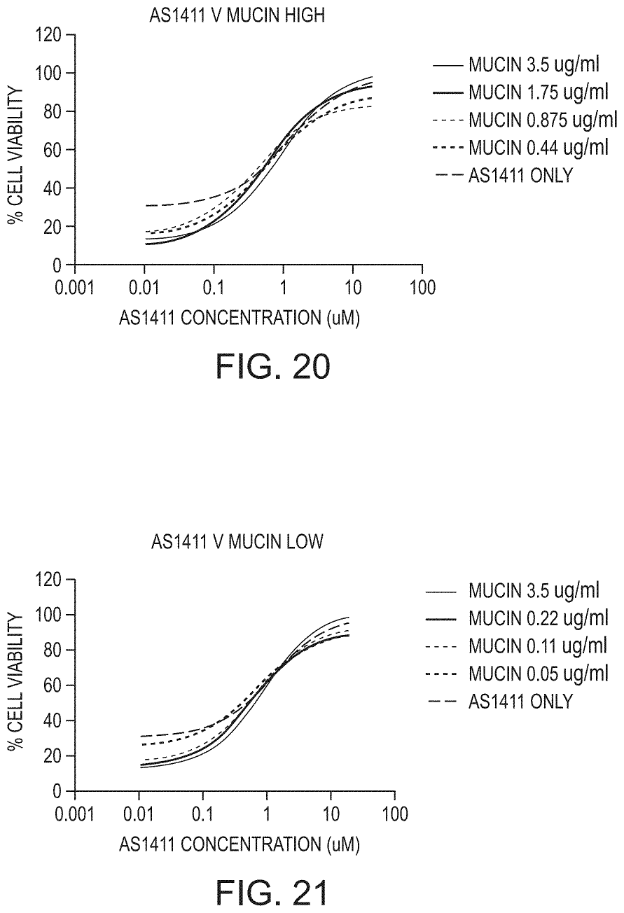

FIGS. 20 and 21 show the percentage of cell viability for varying concentrations of AS1411 and various concentrations of Mucin. Table 5 below shows the EC50 of each Mucin concentration in combination with AS1411. The EC50 is fairly consistent across the different Mucin concentrations.

TABLE 5

| Mucin (ug/ml) + AS1411 | EC50 |
|---|---|
| 3.5 | 0.91 |
| 1.75 | 0.56 |
| 0.875 | 0.40 |
| 0.4375 | 0.62 |
| 0.21875 | 0.61 |
| 0.109375 | 0.65 |
| 0.0546875 | 0.66 |

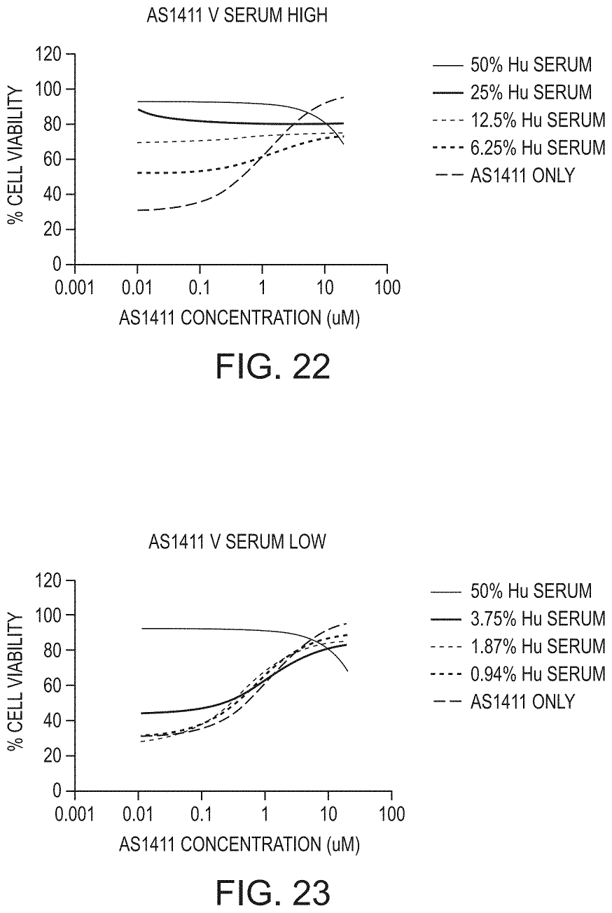

FIGS. 22 and 23 show the percentage of cell viability for varying concentrations of AS1411 and various concentrations of human serum. Table 6 below shows the EC50 of each serum concentration in combination with AS1411. The human serum is a commercially available human serum from INNOVATIVE RESEARCH™ from a COVID-19 naive individual.

TABLE 6

| Serum (%) + AS1411 | EC50 |
|---|---|
| 50 | *Al |
| 25 | *Al |
| 12.5 | 0.55 |
| 6.25 | 1.43 |
| 3.125 | 1.16 |
| 1.5625 | 0.44 |
| 0.78125 | 0.72 |

*Al denotes interference with the virological assay by the serum

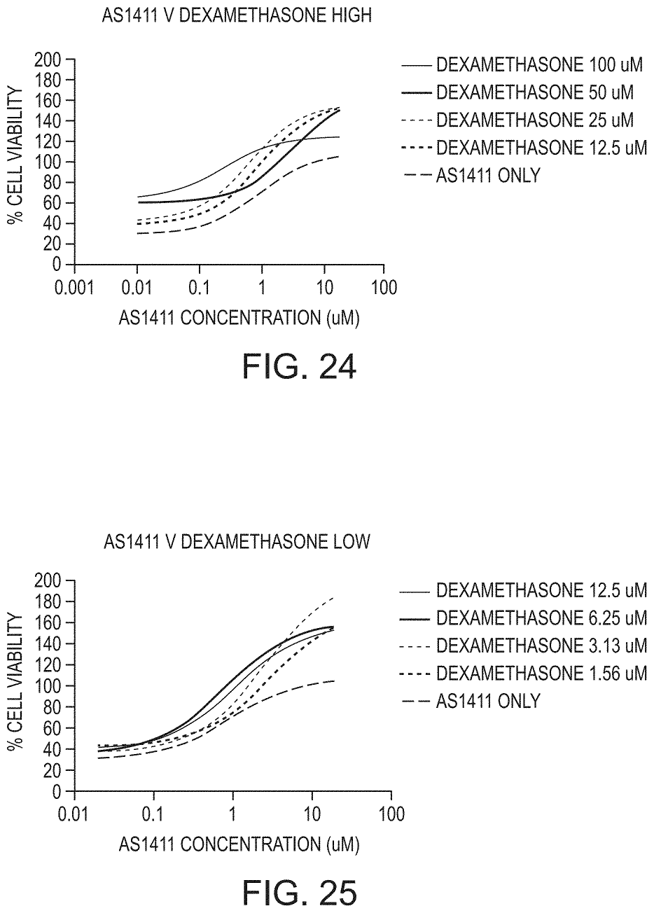

FIGS. 24 and 25 show the percentage of cell viability for varying concentrations of AS1411 and various concentrations of dexamethasone. Table 7 below shows the EC50 of each dexamethasone concentration in combination with AS1411.

TABLE 7

| Dexamethasone (uM) + AS1411 | EC50 |
|---|---|
| 100 | 0.14 |
| 50 | 1.47 |
| 25 | 0.81 |
| 12.5 | 0.83 |
| 6.25 | 0.70 |
| 3.13 | 1.03 |
| 1.56 | 1.42 |

The results of the cell viability experiment show that AS1411 alone is effective for increasing the cell viability, when the cells are challenged with the SARS-CoV-2 virus. AS1411 in combination with other treatments may increase the percentage of cell viability.

### Example 6: EPIAIRWAY™ Tissue Study of AS1411 Alone and in Combination with Other Therapeutically Effective Compounds

The effects of AS1411 alone and in combination with other treatments for COVID-19 were tested by measuring the amount of virus present in EPIAIRWAY™ tissue after administering the SARS-CoV-2 virus at multiplicity of infection (MOI) of 0.1. Progeny virus release was determined by determining the 50% tissue culture infective dose (TCID₅₀) for EPIAIRWAY™ tissue. The amount of virus that was present in the tissue cultures after a period of time was quantified, and is referred to by TCID₅₀/ml. A higher TCID₅₀/ml level indicates that more virus was present in the tissue sample. In this example the treatments were administered 4 hours prior to administration of the virus to the EPIAIRWAY™ tissue. The cytotoxicity of AS1411 by itself was tested with the EPIAIRWAY™ model to demonstrate that AS1411 was not responsible for killing cells. An EPIAIRWAY™ toxicity experiment showed that AS1411 was not toxic across the entirety of tested concentrations, from 0.1 μm to 100 μm.

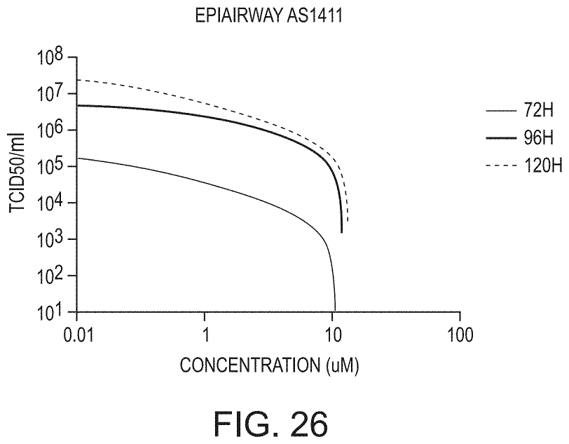

FIG. 26 shows that the amount of virus, as indicated by the TCID₅₀/ml, is lower for tissue samples that received higher doses of AS1411. Table 8 shows the EC50 of AS1411 for the different time periods measured from from the virus challenge to the end of the experiment.

TABLE 8

| Hours Post-Infection | EC50 |
|---|---|
| 72 | 0.9418 |
| 96 | 1.4105 |
| 120 | 0.6497 |
| Average | 1.0001 |

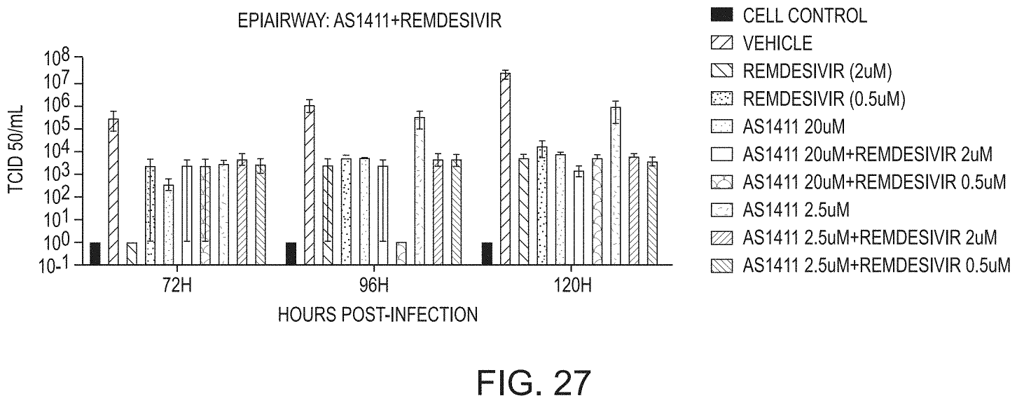

FIG. 27 shows the effectiveness of AS1411 in combination with remdesivir at various concentrations. The error bars on the graph show the standard error of the mean. The cell control group did not receive the virus. The vehicle group received the virus and PBS only. The data demonstrate that AS1411 and remdesivir do not interfere with each other. The data also show that the AS1411 has a dose-dependent effect for the 96-hour and 120-hour periods.

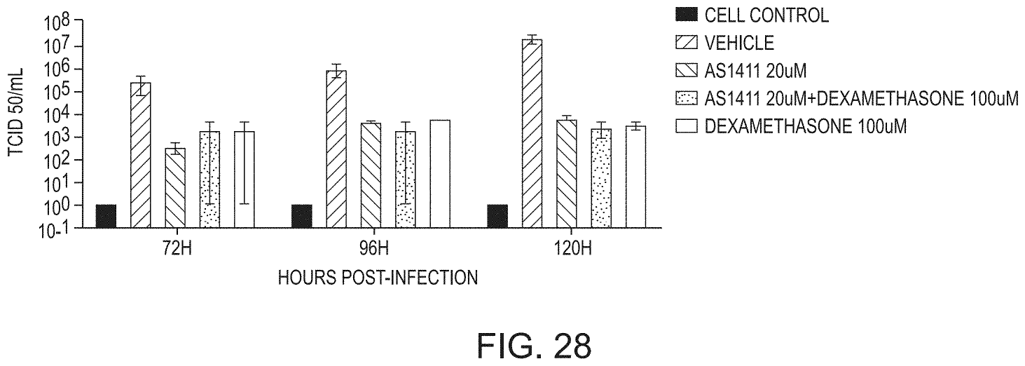

FIG. 28 shows the effectiveness of AS1411 in combination with dexamethasone at various concentrations. The data demonstrate that AS1411 and dexamethasone do not interfere with each other. The effectiveness of dexamethasone alone may be an artifact since the EPIAIRWAY™ tissue does not contain immune cells.

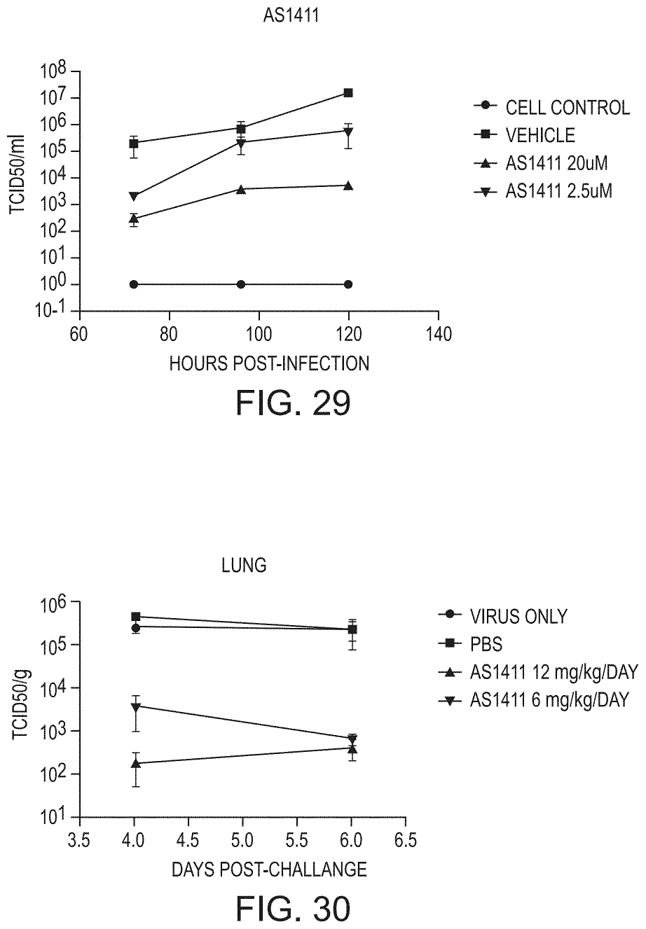

FIG. 29 shows the effectiveness of the low and high concentrations of AS1411 on EPIAIRWAY™ tissue that has been challenged with SARS-CoV-2 virus. The vehicle group received the virus only. The protective effect of the 2.5 μM and 20 μM is demonstrated by the reduction of virus in the AS1411 groups compared to the vehicle group.

### Example 7: TCID₅₀/ml of Tissues from Infected K18-hACE2 Mice

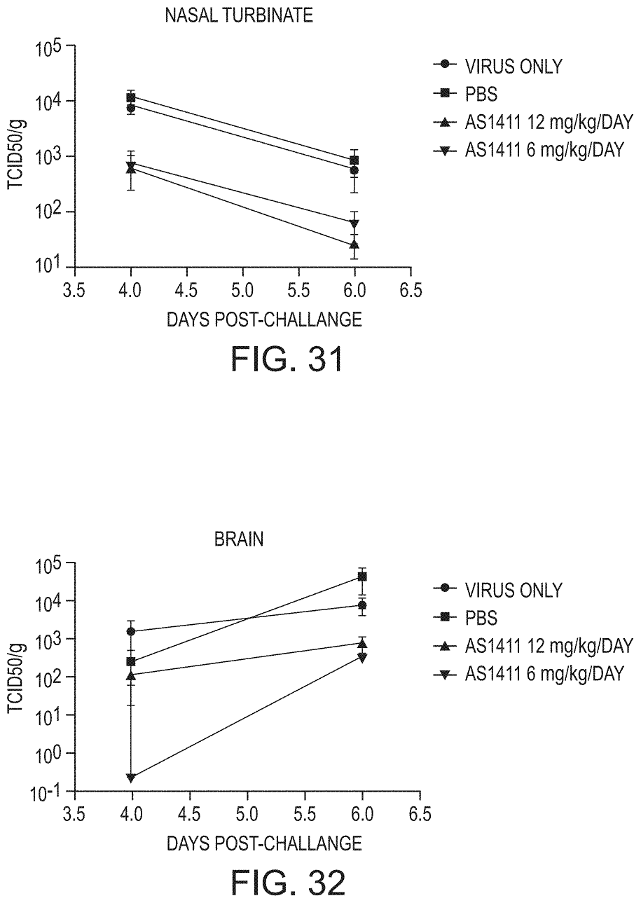

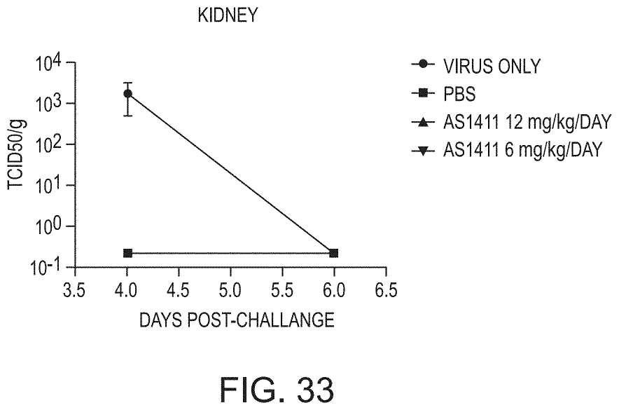

A study of the virus concentration in tissue was carried out on groups of K18-hACE2 mice that were infected with the SARS-CoV-2 virus. FIG. 30-33 show the TCID₅₀/ml from various tissues for 4 cohort groups. The 4 groups are virus only, PBS, 12 mg/kg/per day of AS1411, and 6 mg/kg/per day of AS1411. The administration of the treatments was the same as described in Example 4. The mice were sacrificed at either 4 days post-challenge or 6 days post-challenge. Each group contained 4 mice. FIG. 30 shows that AS1411 was effective for inhibiting the virus in the lungs of the mice. FIGS. 31 and 33 show that AS1411 was effective for inhibiting the virus in the nasal passage and kidneys of the mice, although not as effectively as in the lungs. FIG. 32 shows that AS1411 was not effective for inhibiting the virus in the brain. This is likely due to the difficulty of AS1411 to cross the blood-brain barrier and reach brain tissue. No significant quantity of virus was detected in heart or intestine.

### REFERENCES

[1] Bates et al., International Application, International Publication Number WO 2012/167173, entitled “ANTI-NUCLEOLIN AGENT-CONJUGATED NANOPARTICLES”, filed 1 Jun. 2012.
[2] Bates, et al. “G-quadruplex oligonucleotide AS1411 as a cancer-targeting agent: Uses and mechanisms” Biochica et Biophysica Acta 1861 (2017) pp. 1414-1428.
[3] Bates P J, Miller D M, Trent J O, Xu X, “A New Method for the Diagnosis and Prognosis of Malignant Diseases” International Application, Int'l Pub. No. WO 03/086174 A2 (23 Oct. 2003).
[4] Bates P J, Miller D M, Trent J O, Xu X, “Method for the Diagnosis and Prognosis of Malignant Diseases” U.S. Patent App. Pub., Pub. No. US 2005/0053607 A1 (10 Mar. 2005).
[5] Gonzalez, et al. “Use of Aptamers as Diagnostics Tools and Antibiral Agents for Human Viruses” Pharmaceuticals 9(78): 1-34 (2016).
[6] Bates P J, Kahlon J B, Thomas S D, Trent J O, Miller D M, “Antiproliferative , Activity of G-rich Oligonucleotides Correlates with Protein Binding” J. Biol. Chem. 274:26369-77 (1999).
[7] Miller D M, Bates P J, Trent J O, Xu X, “Method for the Diagnosis and Prognosis of Malignant Diseases” U.S. Patent App. Pub., Pub. No. US 2003/0194754 A1 (16 Oct. 2003).
[8] Bandman O, Yue H, Corley N C, Shah P, “Human Nucleolin-like Protein” U.S. Pat. No. 5,932,475 (3 Aug. 1999).
[9] Reyes-Reyes E M, Teng Y, Bates P J, “A New Paradigm for Aptamer Therapeutic AS1411 Action: Uptake by Macropinocytosis and Its Stimulation by a Nucleolin-Dependent Mechanism” Cancer Res 70(21): 8617-29 (2010).
[10] Mastrangelo et al. “Targeting Host Cell Surface Nucleolin for RSV Therapy: Challenges and Opportunities” Vaccines 5(27): 1-13 (2017).
[11] Chen et al. “Interaction of the Coronavirus Nucleoprotein with Nucleolar Antigens and the Host Cell” J. Virology 76(10): 5233-5250 (May 2002).
[12] U.S. Pat. No. 9,452,219 to Bates et al. (2016).
[13] Myrdal et al. “Advances in Metered Dose Inhaler Technology: Formulation Development” AAPS PharmSciTech, Vol. 15, No. 2, pg. 434-455 (2014).
[14] Mehta et al. “Imagine the Superiority of Dry Powder Inhalers from Carrier Engineering” Journal of Drug Delivery, Volume 2018, Article ID 5635010, 19 pages (2018).
[15] Telkco et al. “Dry Powder Inhaler Formulation” RESPIRATORY CARE, VOL 50 NO 9 (2005).
[16] Baranowski et al. “Ophthalmic Drug Dosage Forms: Characterisation and Research Methods” The ScientificWorld Journal, Volume 2014, Article ID 861904, 14 pages (2014).
[17] Santosh Thorat “Formulation and Product Development of Nasal Spray: An Overview” Scholars Journal of Applied Medical Sciences (SJAMS), 4(8D):2976-2985 (2016).
[18] Geller et al. “Development of an Inhaled Dry-Powder Formulation of Tobramycin Using PulmoSphere Technology” JOURNAL OF AEROSOL MEDICINE AND PULMONARY DRUG DELIVERY, Volume 24, Number 4 (2011).
[19] Syncytium, Wikipedia, Wikimedia Foundation, last edited Sep. 1, 2020, accessed on Sep. 12, 2020 (en.wikipedia.org/wiki/Syncytium).
[20] Cytopathic effect, Wikipedia, Wikimedia Foundation, last edited Mar. 31, 2020, accessed on Sep. 12, 2020 (en.wikipedia.org/wiki/Cytopathic_effect).
[21] Cooper et al. [2014], Front. Chem. 14, 2, 86.
[22] Alkilany, A. M. and C. J. Murphy, Toxicity and cellular uptake of gold nanoparticles: what we have learned so far? Journal of Nanoparticle Research, 2010. 12(7): p. 2313-2333.
[23] Moreau et al., “Evaluation of K18-hACE2 Mice as a Model of SARS-CoV-2 Infection”, Am. J. Trop. Med. Hyg., 103(3), pp. 1215-1219 (2020).
[24] Service, Robert F., “A call to arms”, Science, Vol. 371, Issue 6534, pp. 1092-1095 (2021).

1. A drug delivery system, comprising:
a metering device for pulmonary, nasal or ocular administration, and
a formulation inside the metering device, wherein the formulation comprises an anti-nucleolin agent.

2. (canceled)

3. A method of protecting a mucus membrane of a patient, comprising:
administering an anti-nucleolin to the mucus membrane, and
re-administering the anti-nucleolin agent to the mucus membrane.

4. A method of treating or preventing coronavirus infection, comprising administering an effective amount of an anti-nucleolin agent, to a patient in need thereof.

5. A composition for treating or preventing a coronavirus infection, comprising:
a first therapeutically active compound, and
a second therapeutically active compound,
wherein the first therapeutically active compound is an anti-nucleolin agent, and the second therapeutically active compound treats or prevents a coronavirus infection.

6. (canceled)

7. The drug delivery system of claim 1, wherein the formulation further comprises carrier particles, and the anti-nucleolin agent is on the carrier particles.

8. The drug delivery system of claim 1, wherein the formulation further comprises a propellant.

9. The drug delivery system of claim 1, wherein the metering device is a nasal spray device.

10. The drug delivery system of claim 1, wherein the metering device is an eye dropper.

11. The drug delivery system of claim 7, wherein the carrier particles are selected from the group consisting of sugar carriers, cyclodextrins and amino acids.

12. The drug delivery system of claim 8, wherein the propellant is a hydrofluoroalkane (HFA).

13. The drug delivery system of claim 1, wherein the anti-nucleolin agent is conjugated to nanoparticles.

14. The drug delivery system of claim 13, wherein the nanoparticles comprise at least one member selected from the group consisting of metals, elements and oxides.

15. (canceled)

16. The drug delivery system of claim 1, wherein the anti-nucleolin agent comprises an anti-nucleolin oligonucleotide.

17. The drug delivery system of claim 1, wherein the anti-nucleolin agent comprises an antibody.

18. The drug delivery system of claim 1, wherein the anti-nucleolin agent comprises a nucleolin targeting protein.

19. (canceled)

20. (canceled)

21. The drug delivery system of claim 1, wherein the anti-nucleolin agent is AS1411.

22-28. (canceled)

29. The drug delivery system of claim 1, wherein the coronavirus is SARS-CoV-2.

30. (canceled)

31. The drug delivery system of claim 1, wherein the anti-nucleolin agent is AS1411 and PEG conjugated to gold nanoparticles.

32. (canceled)

33. The composition of claim 5, wherein the second therapeutically active compound is an anti-viral compound and/or an anti-ARDS compound.

34. The composition of claim 5,
wherein the second therapeutically active compound comprises an anti-viral compound, and
the anti-viral compound is selected from the group consisting of remdesivir, convalescent plasma or serum, interferons, baricitinib, masitinib and combinations thereof.

35. The composition of claim 5,
wherein the second therapeutically active compound comprises an anti-ARDS compound, and
the anti-ARDS compound is selected from the group consisting of dexamethasone, methylprednisolone, famotidine, famotidine and cetirizine, anti-inflammatory antibodies and combinations thereof,

36. (canceled)

37. The method of claim 4, wherein the anti-nucleolin agent is administered systematically or to a mucus membrane of the patient.

38. The method of claim 4, further comprising administering a second therapeutically active compound, wherein the second therapeutically active compound treats or prevents a coronavirus infection.


SEQUENCE LISTING (38 sequences)

SEQ ID NO: 1 - DNA, 29 bp, Artificial Sequence
  Description of Artificial Sequence Synthetic polynucleotide sequence
tttggtggtggtggttgtggtggtggtgg

SEQ ID NO: 2 - DNA, 29 bp, Artificial Sequence
  Description of Artificial Sequence Synthetic polynucleotide sequence
tttggtggtggtggttttggtggtggtgg

SEQ ID NO: 3 - DNA, 29 bp, Artificial Sequence
  Description of Artificial Sequence Synthetic polynucleotide sequence
tttggtggtggtggtggtggtggtggtgg

SEQ ID NO: 4 - DNA, 29 bp, Artificial Sequence
  Description of Artificial Sequence Synthetic polynucleotide sequence
tttggtggtggtggtttgggtggtggtgg

SEQ ID NO: 5 - DNA, 13 bp, Artificial Sequence
  Description of Artificial Sequence Synthetic polynucleotide sequence
tggtggtggtggt

SEQ ID NO: 6 - DNA, 14 bp, Artificial Sequence
  Description of Artificial Sequence Synthetic polynucleotide sequence
ggtggttgtggtgg

SEQ ID NO: 7 - DNA, 15 bp, Artificial Sequence
  Description of Artificial Sequence Synthetic polynucleotide sequence
gttgtttggggtggt

SEQ ID NO: 8 - DNA, 15 bp, Artificial Sequence
  Description of Artificial Sequence Synthetic polynucleotide sequence
ttggggggggtgggt

SEQ ID NO: 9 - DNA, 25 bp, Artificial Sequence
  Description of Artificial Sequence Synthetic polynucleotide sequence
ggttggggtgggtggggtgggtggg

SEQ ID NO: 10 - DNA, 26 bp, Artificial Sequence
  Description of Artificial Sequence Synthetic polynucleotide sequence
ggtggtggtggttgtggtggtggtgg

SEQ ID NO: 11 - DNA, 28 bp, Artificial Sequence
  Description of Artificial Sequence Synthetic polynucleotide sequence
tttggtggtggtggttgtggtggtggtg

SEQ ID NO: 12 - DNA, 28 bp, Artificial Sequence
  Description of Artificial Sequence Synthetic polynucleotide sequence
tttggtggtggtggtgtggtggtggtgg

SEQ ID NO: 13 - DNA, 29 bp, Artificial Sequence
  Description of Artificial Sequence Synthetic polynucleotide sequence
ggtggtggtggttgtggtggtggtggttt

SEQ ID NO: 14 - DNA, 32 bp, Artificial Sequence
  Description of Artificial Sequence Synthetic polynucleotide sequence
ggtggttgtggtggttgtggtggttgtggtgg

SEQ ID NO: 15 - DNA, 32 bp, Artificial Sequence
  Description of Artificial Sequence Synthetic polynucleotide sequence
tttggtggtggtggttgtggtggtggtggttt

SEQ ID NO: 16 - DNA, 56 bp, Artificial Sequence
  Description of Artificial Sequence Synthetic polynucleotide sequence
ggtggtggtggttgtggtggtggtggttgtggtggtggtggttgtggtggtggtgg

SEQ ID NO: 17 - DNA, 35 bp, Artificial Sequence
  Description of Artificial Sequence Synthetic polynucleotide sequence
tcgagaaaaactctcctctccttccttcctctcca

SEQ ID NO: 18 - DNA, 29 bp, Artificial Sequence
  Description of Artificial Sequence Synthetic polynucleotide sequence
tttcctcctcctccttctcctcctcctcc

SEQ ID NO: 19 - DNA, 24 bp, Artificial Sequence
  Description of Artificial Sequence Synthetic polynucleotide sequence
ttagggttagggttagggttaggg

SEQ ID NO: 20 - DNA, 11 bp, Artificial Sequence
  Description of Artificial Sequence Synthetic polynucleotide sequence
ggtggtggtgg

SEQ ID NO: 21 - DNA, 14 bp, Artificial Sequence
  Description of Artificial Sequence Synthetic polynucleotide sequence
ggtggttgtggtgg

SEQ ID NO: 22 - DNA, 15 bp, Artificial Sequence
  Description of Artificial Sequence Synthetic polynucleotide sequence
ggttggtgtggttgg

SEQ ID NO: 23 - DNA, 10 bp, Artificial Sequence
  Description of Artificial Sequence Synthetic polynucleotide sequence
gggttttggg

SEQ ID NO: 24 - DNA, 20 bp, Artificial Sequence
  Description of Artificial Sequence Synthetic polynucleotide sequence
ggttttggttttggttttgg

SEQ ID NO: 25 - DNA, 15 bp, Artificial Sequence
  Description of Artificial Sequence Synthetic polynucleotide sequence
ggttggtgtggttgg

SEQ ID NO: 26 - DNA, 12 bp, Artificial Sequence
  Description of Artificial Sequence Synthetic polynucleotide sequence
ggggttttgggg

SEQ ID NO: 27 - DNA, 10 bp, Artificial Sequence
  Description of Artificial Sequence Synthetic polynucleotide sequence
gggttttggg

SEQ ID NO: 28 - DNA, 28 bp, Artificial Sequence
  Description of Artificial Sequence Synthetic polynucleotide sequence
ggggttttggggttttggggttttgggg

SEQ ID NO: 29 - DNA, 24 bp, Artificial Sequence
  Description of Artificial Sequence Synthetic polynucleotide sequence
ttggggttggggttggggttgggg

SEQ ID NO: 30 - DNA, 16 bp, Artificial Sequence
  Description of Artificial Sequence Synthetic polynucleotide sequence
gggtgggtgggtgggt

SEQ ID NO: 31 - DNA, 26 bp, Artificial Sequence
  Description of Artificial Sequence Synthetic polynucleotide sequence
ggttttggttttggttttggttttgg

SEQ ID NO: 32 - DNA, 29 bp, Artificial Sequence
  Description of Artificial Sequence Synthetic polynucleotide sequence
tttcctcctcctccttctcctcctcctcc

SEQ ID NO: 33 - DNA, 26 bp, Artificial Sequence
  Description of Artificial Sequence Synthetic polynucleotide sequence
cctcctcctccttctcctcctcctcc

SEQ ID NO: 34 - DNA, 6 bp, Artificial Sequence
  Description of Artificial Sequence Synthetic polynucleotide sequence
tggggt

SEQ ID NO: 35 - DNA, 7 bp, Artificial Sequence
  Description of Artificial Sequence Synthetic polynucleotide sequence
gcatgct

SEQ ID NO: 36 - DNA, 11 bp, Artificial Sequence
  Description of Artificial Sequence Synthetic polynucleotide sequence
gcggtttgcgg

SEQ ID NO: 37 - DNA, 4 bp, Artificial Sequence
  Description of Artificial Sequence Synthetic polynucleotide sequence
tagg

SEQ ID NO: 38 - DNA, 23 bp, Artificial Sequence
  Description of Artificial Sequence Synthetic polynucleotide sequence
ggggttggggtgtggggttgggg